(19) United States
(12) Patent Application Publication
(10) Pub. No.: US 2018/0114321 A1
(43) Pub. Date: Apr. 26, 2018

(54) DYNAMIC ANALYSIS SYSTEM

(71) Applicant: Konica Minolta, Inc., Tokyo (JP)

(72) Inventor: Noritsugu MATSUTANI, Tokyo (JP)

(21) Appl. No.: 15/783,173

(22) Filed: Oct. 13, 2017

(30) Foreign Application Priority Data
Oct. 21, 2016 (JP) 2016-206480

(51) Int. Cl. G06T 7/00; G06T 7/149

(52) U.S. Cl. CPC G06T 7/0016; A61B 5/0871; G06T 2207/30061; G06T 7/149

(57) ABSTRACT

A dynamic analysis system analyzes a dynamic image of at least one breathing cycle. The dynamic image is obtained by imaging a dynamic state of a chest of a subject. The dynamic analysis system includes a hardware processor. The hardware processor sets a characteristic point on a lung region in the dynamic image, measures a displacement of the characteristic point at each time phase of the dynamic image, obtains a reference ventilation amount of the subject, and calculates a ventilation amount at the each time phase based on the reference ventilation amount and the displacement of the characteristic point at the each time phase.

### CROSS-REFERENCE TO RELATED APPLICATIONS

The present U.S. patent application claims a priority under the Paris Convention of Japanese Patent Application No. 2016-206480 filed on Oct. 21, 2016, the entire disclosure of which, including the description, claims, drawings and abstract, is incorporated herein by reference in its entirety.

### BACKGROUND

### 1. Technological Field

The present invention relates to a dynamic analysis system.

### 2. Description of the Related Art

There has been disclosed a device that analyzes a dynamic image obtained by imaging a dynamic state of a chest so as to provide information effective in diagnosis of a ventilation function of lungs.

For example, there is described in Japanese Patent Application Publication No. 2009-153678 a dynamic analysis system that obtains information on an absolute ventilation amount between the maximal expiratory level and the maximal inspiratory level, calculates an estimated ventilation amount per unit signal change amount from (i) the absolute ventilation amount and (ii) a signal change amount indicating the amount of change in signal value between a frame image at the maximal expiratory level and a frame image at the maximal inspiratory level of a dynamic image, and calculates and provides an estimated ventilation amount at each time phase by multiplying, by a value of the estimated ventilation amount per unit signal change amount, a signal change amount at each time phase indicating the amount of change in signal value at each time phase from the maximal expiratory level or the maximal inspiratory level.

However, a signal change amount(s) contains signal change (noise) due to, for example, movements of artifacts accompanying body movement and/or breathing. Hence, the art described in Japanese Patent Application Publication No. 2009-153678 may not be able to calculate a ventilation amount(s) with high accuracy.

### SUMMARY

Objects of the present invention include calculating, from a dynamic chest image, a ventilation amount(s) with high accuracy.

In order to achieve at least one of the abovementioned objects, according to an aspect of the present invention, there is provided a dynamic analysis system that analyzes a dynamic image of at least one breathing cycle obtained by imaging a dynamic state of a chest of a subject, including: a hardware processor that sets a characteristic point on a lung region in the dynamic image, measures a displacement of the characteristic point at each time phase of the dynamic image, obtains a reference ventilation amount of the subject, and calculates a ventilation amount at the each time phase based on the reference ventilation amount and the displacement of the characteristic point at the each time phase.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows the overall configuration of a dynamic analysis system according to embodiments of the present invention;

FIG. 2 is a flowchart of an imaging control process performed by a hardware processor of an imaging console shown in FIG. 1;

FIG. 3 is a flowchart of a ventilation amount calculation process A performed by a hardware processor of a diagnostic console shown in FIG. 1;

FIG. 4 is a diagram to explain the contour of the bottom part of a lung region and the contour of the outer side part of a lung region;

FIG. 5 shows an example of a ventilation amount calculation result screen; and

FIG. 6 is a flowchart of a ventilation amount calculation process B performed by the hardware processor of the diagnostic console shown in FIG. 1.

### DETAILED DESCRIPTION OF EMBODIMENTS

Hereinafter, one or more embodiments of the present invention will be described with reference to the drawings. However, the scope of the invention is not limited to the disclosed embodiments.

### First Embodiment

### [Configuration of Dynamic Analysis System 100]

First, the configuration of a first embodiment is described.

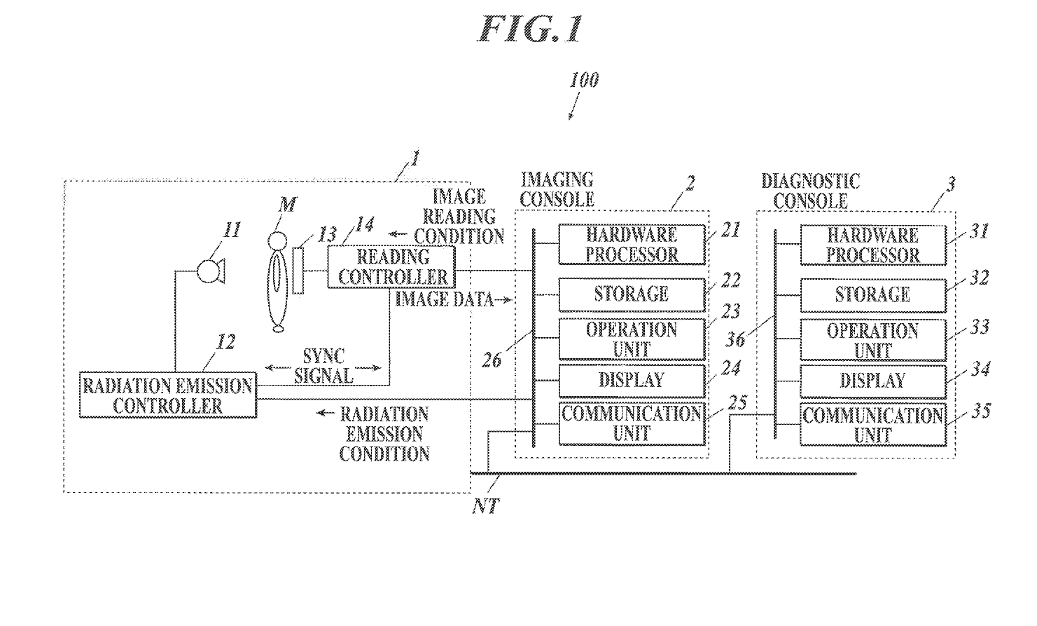

FIG. 1 shows the overall configuration of a dynamic analysis system 100 according to the embodiment(s) of the present invention.

As shown in FIG. 1, the dynamic analysis system 100 includes: an imager 1; an imaging console 2 connected with the imager 1 via a communication cable or the like; and a diagnostic console 3 connected with the imaging console 2 via a communication network NT, such as a LAN (Local Area Network). These components of the dynamic analysis system 100 are in conformity with DICOM (Digital Image and Communications in Medicine) standard and communicate with one another in conformity with DICOM.

### [Configuration of Imager 1]

The imager 1 is an imager that images a cyclic dynamic state of the chest of a subject, for example. Examples of the cyclic dynamic state thereof include: change in shape of the lungs by expansion and contraction of the lungs with breathing; and pulsation of the heart. Dynamic imaging (kinetic imaging) is performed by repeatedly emitting pulsed radiation, such as pulsed X-rays, to a subject at predetermined time intervals (pulse emission) or continuously emitting radiation without a break to a subject at a low dose rate (continuous emission), thereby obtaining a plurality of images showing the dynamic state. A series of images at respective time phases obtained by dynamic imaging is called a dynamic image. Images (images at respective time phases) constituting a dynamic image are called frame images. In the embodiments described hereinafter, dynamic imaging is performed by pulse emission as an example.

A radiation source 11 is disposed to face a radiation detector 13 having a subject M in between, and emits radiation (X-rays) to the subject M under the control of a radiation emission controller 12.

The radiation emission controller 12 is connected with the imaging console 2, and controls the radiation source 11 on the basis of radiation emission conditions input from the imaging console 2 so as to perform radiation imaging. The radiation emission conditions input from the imaging console 2 include a pulse rate, a pulse width, a pulse interval, the number of frames (frame images) to be taken by one imaging, a value of current of an X-ray tube, a value of voltage of the X-ray tube, and a type of added filter. The pulse rate is the number of times radiation is emitted per second, and matches the frame rate described below. The pulse width is a period of time for one radiation emission. The pulse interval is a period of time from the start of one radiation emission to the start of the next radiation emission, and matches the frame interval described below.

The radiation detector 13 is constituted of a semiconductor image sensor, such as an FPD. The FPD is constituted of detection elements (pixels) arranged at predetermined points on a substrate, such as a glass substrate, in a matrix. The detection elements detect radiation (intensity of radiation) that has been emitted from the radiation source 11 and passed through at least a subject M, convert the detected radiation into electric signals, and accumulate the electric signals therein. The pixels are provided with switches, such as TFTs (Thin Film Transistors). There are an indirect conversion type FPD that converts X-rays into electric signals with photoelectric conversion element(s) via scintillator(s) and a direct conversion type FPD that directly converts X-rays into electric signals. Either of them can be used.

The radiation detector 13 is disposed to face the radiation source 11 having a subject M in between.

A reading controller 14 is connected with the imaging console 2. The reading controller 14 controls the switches of the pixels of the radiation detector 13 on the basis of image reading conditions input from the imaging console 2 to switch the pixels to read the electric signals accumulated in the pixels, thereby reading the electric signals accumulated in the radiation detector 13 and obtaining image data. This image data is a frame image(s). The reading controller 14 outputs the obtained frame images to the imaging console 2. The image reading conditions include a frame rate, a frame interval, a pixel size, and an image size (matrix size). The frame rate is the number of frame images to be obtained per second, and matches the pulse rate described above. The frame interval is a period of time from the start of one frame image obtaining action to the start of the next frame image obtaining action, and matches the pulse interval described above.

The radiation emission controller 12 and the reading controller 14 are connected to each other, and exchange sync signals so as to synchronize radiation emission actions with image reading actions.

### [Configuration of Imaging Console 2]

The imaging console 2 outputs the radiation emission conditions and the image reading conditions to the imager 1 so as to control the radiation imaging and the radiation image reading actions performed by the imager 1, and also displays the dynamic image obtained by the imager 1 so that a radiographer, such as a radiologist, can check if positioning has no problem, and also can determine if the dynamic image is suitable for diagnosis.

The imaging console 2 includes, as shown in FIG. 1, a hardware processor 21, a storage 22, an operation unit 23, a display 24 and a communication unit 25. These components are connected to one another via a bus 26.

The hardware processor 21 includes a CPU (Central Processing Unit) and a RAM (Random Access Memory). The CPU of the hardware processor 21 reads a system program and various process programs stored in the storage 22 in response to operation on the operation unit 23, opens the read programs in the RAM, and performs various processes, such as the below-described imaging control process, in accordance with the opened programs, thereby performing concentrated control of actions of the components of the imaging console 2 and the radiation emission actions and the reading actions of the imager 1.

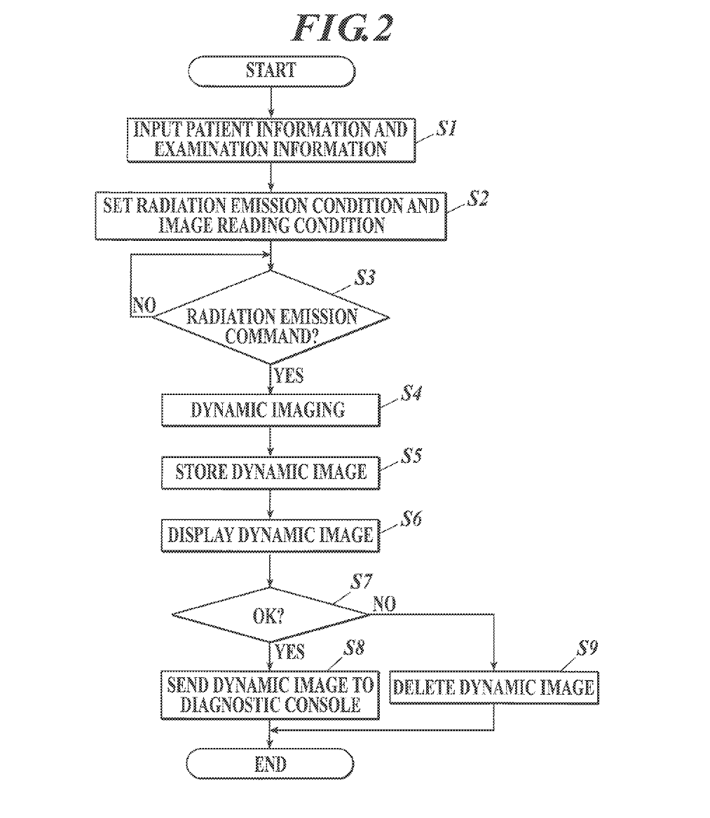

The storage 22 is constituted of a nonvolatile semiconductor memory, a hard disk or the like. The storage 22 stores therein various programs to be executed by the hardware processor 21, parameters necessary to perform processes of the programs, data, such as process results, and so forth. For example, the storage 22 stores therein a program for the imaging control process shown in FIG. 2. The storage 22 also stores therein the radiation emission conditions and the image reading conditions for respective imaging sites. The programs are stored in the form of a computer readable program code(s), and the hardware processor 21 acts in accordance with the program code.

The operation unit 23 includes: a keyboard including cursor keys, number input keys and various function keys; and a pointing device, such as a mouse, and outputs, to the hardware processor 21, command signals input by key operation on the keyboard or by mouse operation. The operation unit 23 may have a touchscreen on the display screen of the display 24. In this case, the operation unit 23 outputs command signals input via the touchscreen to the hardware processor 21.

The display 24 is constituted of a monitor, such as an LCD (Liquid Crystal Display) or a CRT (Cathode Ray Tube), and displays thereon commands input from the operation unit 23, data and so forth in accordance with commands of display signals input from the hardware processor 21.

The communication unit 25 includes a LAN adapter, a modem and a TA (Terminal Adapter), and controls data exchange with devices connected to the communication network NT.

### [Configuration of Diagnostic Console 3]

The diagnostic control 3 is a dynamic analyzer that obtains the dynamic image from the imaging console 2, analyzes the dynamic image, and displays the obtained dynamic image and/or the analysis result of the dynamic image to help a doctor(s) make a diagnosis. The diagnostic console 3 includes, as shown in FIG. 1, a hardware processor 31, a storage 32, an operation unit 33, a display 34 and a communication unit 35. These components are connected to one another via a bus 36.

The hardware processor 31 includes a CPU and a RAM. The CPU of the hardware processor 31 reads a system program and various process programs stored in the storage 32 in response to operation on the operation unit 33, opens the read programs in the RAM, and performs various processes, such as the below-described ventilation amount calculation process A, in accordance with the opened programs, thereby performing concentrated control of actions of the components of the diagnostic console 3.

The storage 32 is constituted of a nonvolatile semiconductor memory, a hard disk or the like. The storage 32 stores therein various programs, including a program for the ventilation amount calculation process A, to be executed by the hardware processor 31, parameters necessary to perform processes of the programs, data, such as process results, and so forth. The programs are stored in the form of a computer readable program code(s), and the hardware processor 31 acts in accordance with the program code.

The storage 32 also stores therein body thicknesses for physical characteristics, such as heights, weights, ages and sexes.

The operation unit 33 includes: a keyboard including cursor keys, number input keys and various function keys; and a pointing device, such as a mouse, and outputs, to the hardware processor 31, command signals input by key operation on the keyboard or by mouse operation. The operation unit 33 may have a touchscreen on the display screen of the display 34. In this case, the operation unit 33 outputs command signals input via the touchscreen to the hardware processor 31.

The display 34 is constituted of a monitor, such as an LCD or a CRT, and performs various types of display in accordance with commands of display signals input from the hardware processor 31.

The communication unit 35 includes a LAN adapter, a modem and a TA, and controls data exchange with devices connected to the communication network NT.

### [Actions of Dynamic Analysis System 100]

Next, actions of the dynamic analysis system 100 are described.

### [Actions of Imager 1 and Imaging Console 2]

First, imaging actions performed by the imager 1 and the imaging console 2 are described.

FIG. 2 shows the imaging control process performed by the hardware processor 21 of the imaging console 2. The imaging control process is performed by the hardware processor 21 in cooperation with the program stored in the storage 22.

First, a radiographer operates the operation unit 23 of the imaging console 2 so as to input patient information (patient name, height, weight, age, sex, etc.) on an examinee (subject M), and examination information (an imaging site (here, the chest), a direction (here, from the front), a breathing style (chest breathing or abdominal breathing), etc.) on an examination to be performed on the examinee (Step S1).

Next, the hardware processor 21 reads radiation emission conditions from the storage 22 so as to set them in the radiation emission controller 12, and also reads image reading conditions from the storage 22 so as to set them in the reading controller 14 (Step S2).

Next, the hardware processor 21 waits for a radiation emission command to be input by radiographer operation on the operation unit 23 (Step S3). Here, the radiographer places the subject M between the radiation source 11 and the radiation detector 13 and performs positioning. Further, in the embodiment(s), because imaging is performed during breathing, the radiographer instructs the examinee (subject M) to relax and encourages him/her to do quiet breathing, or may lead the examinee to deep breathing by saying “Breathe in.”, “Breathe out.” and so forth. When preparations for imaging are complete, the radiographer operates the operation unit 23 so as to input the radiation emission command.

When receiving the radiation emission command input through the operation unit 23 (Step S3; YES), the hardware processor 21 outputs an imaging start command to the radiation emission controller 12 and the reading controller 14 to start dynamic imaging (Step S4). That is, the radiation source 11 emits radiation at pulse intervals set in the radiation emission controller 12, and accordingly the radiation detector 13 obtains (generates) a series of frame images.

When imaging for a predetermined number of frame images finishes, the hardware processor 21 outputs an imaging end command to the radiation emission controller 12 and the reading controller 14 to stop the imaging actions. The number of frame images to be taken covers at least one breathing cycle.

The frame images obtained by imaging are successively input to the imaging console 2 and stored in the storage 22, the frame images being correlated with respective numbers indicating what number in the imaging order the respective frame images have been taken (frame numbers) (Step S5), and also displayed on the display 24 (Step S6). The radiographer checks the positioning or the like with the displayed dynamic image, and determines whether the dynamic image obtained by dynamic imaging is suitable for diagnosis (Imaging OK) or re-imaging is necessary (Imaging NG). Then, the radiographer operates the operation unit 23 so as to input the determination result.

When the determination result “Imaging OK” is input by the radiographer performing a predetermined operation on the operation unit 23 (Step S7; YES), the hardware processor 21 attaches, to the respective frame images obtained by dynamic imaging (e g writes, in the header region of the image data in DICOM), information such as an ID to identify the dynamic image, the patient information, the examination information, the radiation emission conditions, the image reading conditions, and the respective numbers indicating what number in the imaging order the respective frame images have been taken (frame numbers), and sends the same to the diagnostic console 3 through the communication unit 25 (Step S8), and then ends the imaging control process. On the other hand, when the determination result “Imaging NG” is input by the radiographer performing a predetermined operation on the operation unit 23 (Step S7; NO), the hardware processor 21 deletes the frame images (the series of frame images) from the storage 22 (Step S9), and then ends the imaging control process. In this case, re-imaging is necessary.

### [Actions of Diagnostic Console 3]

Next, actions of the diagnostic console 3 are described.

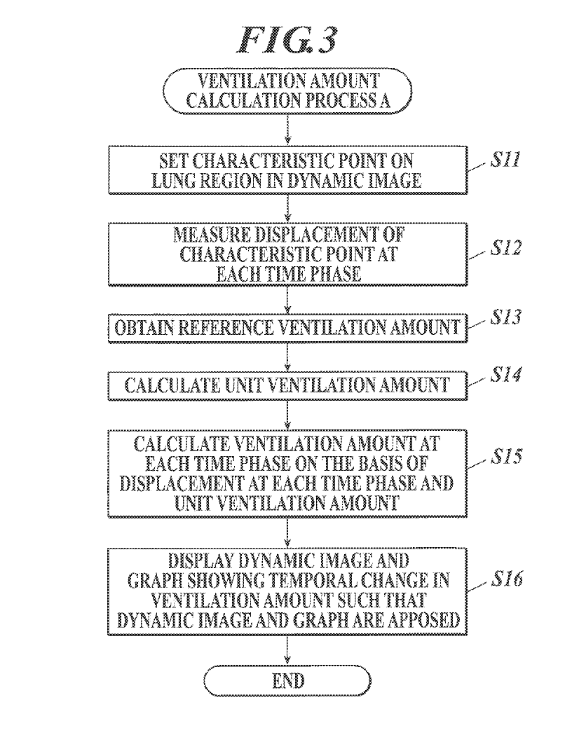

In the diagnostic console 3, when receiving a series of frame images of a dynamic image from the imaging console 2 through the communication unit 35, the hardware processor 31 performs the ventilation amount calculation process A shown in FIG. 3 in cooperation with the program stored in the storage 32. Note that the horizontal direction and the vertical direction of each frame image of the dynamic image are the X direction and the Y direction, respectively.

Hereinafter, the flow of the ventilation amount calculation process A is described with reference to FIG. 3.

First, the hardware processor 31 sets a characteristic point at one point on lung regions (i.e. one characteristic point on one of the right and left lung regions) in the dynamic image (Step S11).

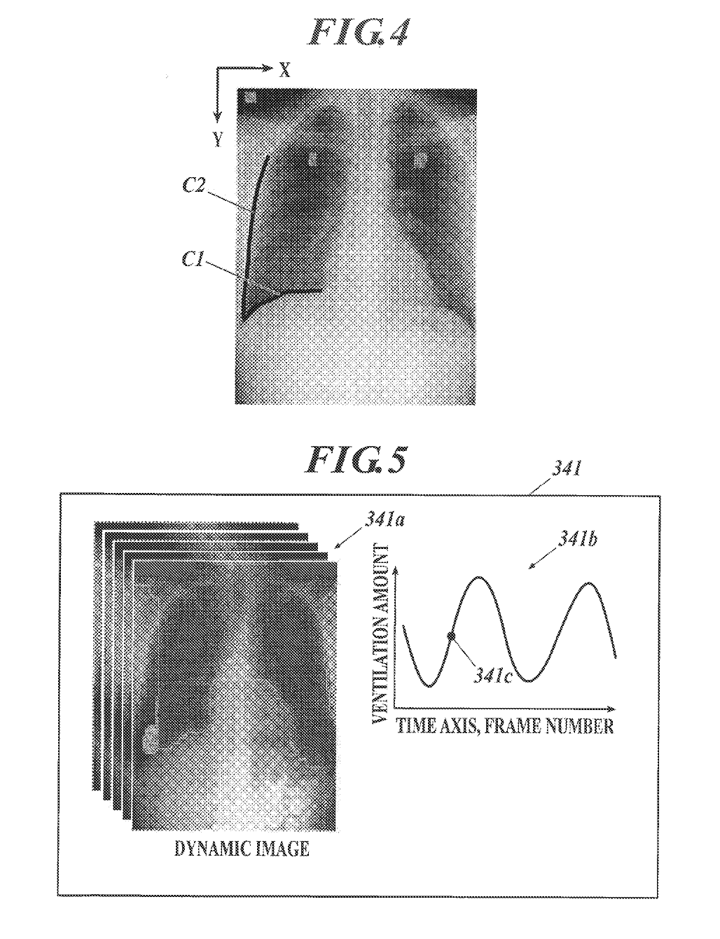

In abdominal breathing, ventilation is carried out mainly by movement of the diaphragm in the up-down direction and accordingly movement of the bottom parts of the lung fields (the right and left lung fields) in the up-down direction. Meanwhile, in chest breathing, ventilation is carried out mainly by expansion and contraction of the thorax and accordingly expansion and contraction of the lung fields in the outside direction and the inside direction, respectively. Hence, for example, in the case of abdominal breathing, it is preferable to set a characteristic point on the contour of the bottom part of a lung region (C1 in FIG. 4), whereas in the case of chest breathing, it is preferable to set a characteristic point on the contour of the outer side part of a lung region (C2 in FIG. 4), in particular, of the right lung region because the left lung field has a part that overlaps the heart. In this embodiment, the characteristic point is set on the right lung region.

The characteristic point may be set manually by a user, or may be set automatically by the hardware processor 31.

If the characteristic point is set manually by a user, for example, in Step S11, one (e.g. the 1^{st} frame image) of the frame images of the dynamic image is displayed on the display 34, and a point on a lung region in the displayed frame image specified (pressed) with the operation unit 33 is set as the characteristic point.

If the characteristic point is set automatically, a lung region is extracted from one of the frame images of the dynamic image, and the characteristic point is set, for example, in the case of abdominal breathing, on the contour of the bottom part of the extracted lung region (C1 in FIG. 4), and in the case of chest breathing, on the contour of the outer side part of the extracted lung region (C2 in FIG. 4). Any method can be used for extraction of the lung region. For example, a threshold value is obtained from a histogram of pixel signal values of the frame image, in which the lung region is to be recognized, by discriminant analysis, and a region having a higher signal value(s) than the threshold value is extracted as a lung region candidate. Then, edge detection is performed on around the border of the extracted lung region candidate, and, in small regions around the border, points where the edge is the maximum are extracted along the border, so that the border of the lung region can be extracted.

Next, the hardware processor 31 measures a displacement of the characteristic point at each time phase (Step S12).

More specifically, first, the position of the characteristic point is measured from the respective frame images at the respective time phases of the dynamic image. For example, if the characteristic point is set on the contour of the bottom part of a lung region in Step S11, as described above, the lung region (the right lung region in this embodiment), on which the characteristic point is set, is extracted from each frame image, and the position of a point that is on the contour of the bottom part of the extracted lung region and has the same x coordinate as that of the characteristic point set in Step S11 is obtained as the position of the characteristic point in the frame image. Alternatively, for example, if the characteristic point is set on the contour of the outer side part of a lung region in Step S11, as described above, the lung region, on which the characteristic point is set, is extracted from each frame image, and the position of a point that is on the contour of the outer side part of the extracted lung region and has the same y coordinate as that of the characteristic point set in Step S11 is obtained as the position of the characteristic point in the frame image.

Then, the relative distance from the position of the characteristic point in a frame image at the maximal expiratory level to the position of the characteristic point at each time phase is calculated as the displacement of the characteristic point at each time phase. The frame image at the maximal expiratory level is, if the characteristic point is set on the contour of the bottom part of a lung region, the frame image in which the position of the characteristic point is the highest in the vertical direction, and if the characteristic point is set on the contour of the outer side part of a lung region, the frame image in which the position of the characteristic point is the innermost in the horizontal direction.

Next, the hardware processor 31 obtains a reference ventilation amount (Step S13).

For example, if the characteristic point is set on the contour of the bottom part of a lung region, the reference ventilation amount can be calculated on the basis of the maximum displacement of the characteristic point among the displacements thereof calculated in Step S12 by the following Formula 1.

Reference Ventilation Amount=Maximum Displacement×Body Thickness×Width of Lung Fields [Formula 1]

As the body thickness, the body thickness that is stored in the storage 32 for the physical characteristics of the examinee identified by the patient information attached to the dynamic image is used. The width of the lung fields is a value measured from the dynamic image; for example, the mean of distances from the outer side of the right lung region to the outer side of the left lung region in the respective frame images. Where in the vertical direction of the lung fields the width of the lung fields is measured may be specified by a user with the operation unit 33. Alternatively, the width at the middle in the vertical direction of the lung regions or the widest width thereof may be measured automatically.

For example, if the characteristic point is set on the contour of the outer side part of a lung region, the reference ventilation amount can be calculated on the basis of the maximum displacement of the characteristic point among the displacements thereof calculated in Step S12 by the following Formula 2.

Reference Ventilation Amount=Maximum Displacement×Body Thickness×Height of Lung Fields [Formula 2]

As the body thickness, the body thickness that is stored in the storage 32 for the physical characteristics of the examinee identified by the patient information attached to the dynamic image is used. The height of the lung fields is a value measured from the dynamic image; for example, the mean of distances from the apex of the right lung region to the bottom of the right lung region in the respective frame images.

By either of the above Formula 1 and Formula 2, a volume change amount of the lung fields by breathing can be calculated, and this volume change amount is obtained as the reference ventilation amount.

If the dynamic image contains a plurality of breathing cycles, the maximum displacement of the characteristic point is calculated for each breathing cycle, and the reference ventilation amount is calculated by the above Formula 1 or Formula 2, taking the mean of the maximum displacements in the respective breathing cycles as the “Maximum Displacement” in the Formula 1 or Formula 2. This can absorb variation in the ventilation amount among the breathing cycles.

The method for obtaining the reference ventilation amount is not limited to the above, and hence, for example, the maximum ventilation amount of the subject M measured with a spirometer may be obtained as the reference ventilation amount.

Next, the hardware processor 31 calculates a unit ventilation amount that indicates a ventilation amount per unit displacement (Step S14). The unit ventilation amount can be calculated, for example, by the following Formula 3.

Unit Ventilation Amount=Reference Ventilation Amount÷Maximum Displacement [Formula 3]

Next, the hardware processor 31 calculates a ventilation amount at each time phase (Step S15).

The ventilation amount at each time phase can be calculated by the following Formula 4.

Ventilation Amount=Unit Ventilation Amount×Displacement of Characteristic Point [Formula 4]

Thus, the ventilation amount at each time phase is calculated on the basis of the displacement of the characteristic point at each time phase. This can reduce influence of noise, so that the ventilation amount can be calculated with high accuracy.

Next, the hardware processor 31 displays the calculation result of the ventilation amount on the display 34 (Step S16), and ends the ventilation amount calculation process A.

FIG. 5 shows an example of a ventilation amount calculation result screen 341 displayed in Step S16. As shown in FIG. 5, on the ventilation amount calculation result screen 341, a dynamic image 341a and a graph 341b showing temporal change in the ventilation amount are displayed side by side. On the graph 341b, a mark (e.g. an annotation) 341c indicating the position of the time phase of the displayed frame image of the dynamic image 341a is displayed in sync with the dynamic image 341a.

Thus, the dynamic image 341a and the graph 341b are synchronized with each other and displayed in such a way as to be apposed. This allows a doctor(s) to observe the dynamic image 341a while checking the ventilation amount, and therefore can improve diagnostic efficiency.

In the above, one characteristic point is set on the whole of the lung regions. Alternatively, a plurality of characteristic points may be set thereon. In this case, the displacement of each characteristic point is calculated, and the ventilation amount is calculated by substituting the mean of the displacements of the respective characteristic points for the “Displacement of Characteristic Point” in the above Formula 4. There is a case where the displacement of a characteristic point slightly differs according to the position of the characteristic point because of individual difference in body structure and/or imaging environment. Use of the mean of displacements of multiple characteristic points can absorb the difference in the displacement, so that the ventilation amount can be calculated with higher accuracy.

### Second Embodiment

Next, a second embodiment of the present invention is described.

In the first embodiment, the ventilation amount of the whole of the lungs (the lung fields) is calculated. Alternatively, in the second embodiment, the ventilation amounts of the right and left lungs (the right and left lung fields) are calculated, respectively.

The configuration and the imaging actions of the dynamic analysis system 100 in the second embodiment are the same as those in the first embodiment. Hence, descriptions thereof are not repeated here, and hereinafter, actions of the diagnostic console 3 in the second embodiment are described.

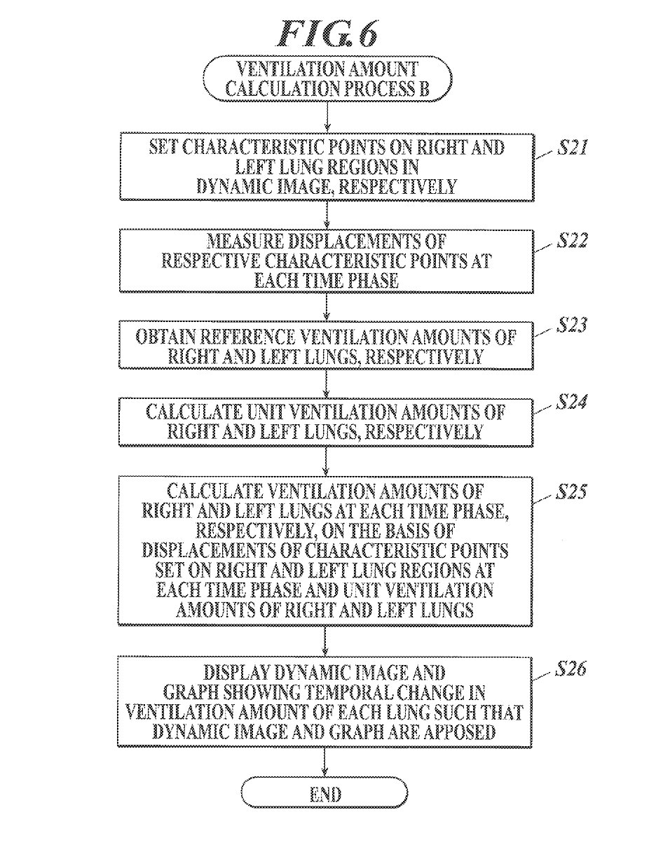

FIG. 6 is a flowchart of a ventilation amount calculation process B in the second embodiment. In the diagnostic console 3, when receiving a series of frame images of a dynamic image from the imaging console 2 through the communication unit 35, the hardware processor 31 performs the ventilation amount calculation process B in cooperation with the program stored in the storage 32. Hereinafter, the ventilation amount calculation process B is described with reference to FIG. 6.

First, the hardware processor 31 sets characteristic points on the right and left lung regions in the dynamic image, respectively (Step S21).

The method for setting the characteristic points in Step S21 is the same as that described for Step S1l in FIG. 3 except that the characteristic points are set on the right and left lung regions, respectively, in Step S21. Hence, description thereof is not repeated here.

Next, the hardware processor 31 measures displacements of the respective characteristic points on the right and left lung regions at each time phase (Step S22). Step S22 is the same as Step S12 in FIG. 3. Hence, description thereof is not repeated here.

Next, the hardware processor 31 obtains reference ventilation amounts of the right and left lung fields, respectively (Step S23).

For example, if the characteristic points are set on the contours of the bottom parts of the right and left lung regions, respectively, the reference ventilation amounts of the right and left lung fields can be calculated on the basis of the maximum displacements of the respective characteristic points on the right and left lung regions, respectively, by the following Formula 5. Alternatively, for example, if the characteristic points are set on the contours of the outer side parts of the right and left lung regions, respectively, the reference ventilation amounts of the right and left lung fields can be calculated on the basis of the maximum displacements of the respective characteristic points on the right and left lung regions, respectively, by the following Formula 6.

Reference Ventilation Amount=Maximum Displacement×Body Thickness×Width of Lung Fields±2 [Formula 5]

Reference Ventilation Amount=Maximum Displacement×Body Thickness×Height of Lung Fields±2 [Formula 6]

The body thickness, the width of the lung fields and the height of the lung fields are the same as those described for Step S13 in FIG. 3.

If the dynamic image contains a plurality of breathing cycles, for each of the characteristic points on the right and left lung regions, the maximum displacement of the characteristic point is calculated for each breathing cycle, and the reference ventilation amount is calculated by the above Formula 5 or Formula 6, taking the mean of the maximum displacements in the respective breathing cycles as the “Maximum Displacement” in the Formula 5 or Formula 6. This can absorb variation in the ventilation amount among the breathing cycles.

The method for obtaining the reference ventilation amounts is not limited to the above, and hence, for example, the reference ventilation amounts of the right and left lung fields of the subject M may be obtained by using the maximum ventilation amount of the subject M measured with a spirometer. In this case, weighting coefficients for the right and left lung fields are calculated on the basis of the ratio of the maximum displacements of the characteristic points on the right and left lung regions, and the maximum ventilation amount measured with the spirometer is multiplied by each of the calculated weighting coefficients, so that the reference ventilation amount of the right lung field and the reference ventilation amount of the left lung field are calculated. For example, if the maximum ventilation amount measured with a spirometer is 1 L, the maximum displacement of the characteristic point on the left lung region is 4 cm, and the maximum displacement of the characteristic point on the right lung region is 6 cm, the reference ventilation amount of the left lung field is 400 mL, and the reference ventilation amount of the right lung field is 600 mL.

Next, the hardware processor 31 calculates unit ventilation amounts, each of which indicates a ventilation amount per unit displacement, of the right and left lung fields, respectively (Step S24). The unit ventilation amount of each lung field can be calculated, for example, by the above Formula 3.

Next, the hardware processor 31 calculates ventilation amounts of the right and left lung fields at each time phase, respectively (Step S25).

The ventilation amount of each lung field at each time phase can be calculated by the above Formula 4.

Thus, in the second embodiment, the ventilation amounts of the right and left lung fields at each time phase are calculated on the basis of the displacements of the respective characteristic points on the right and left lung regions at each time phase, respectively. This can reduce influence of noise, so that the ventilation amount of each lung field can be calculated with high accuracy.

Next, the hardware processor 31 displays the calculation results of the ventilation amounts of the right and left lung fields on the display 34 (Step S26), and ends the ventilation amount calculation process B.

The ventilation amount calculation result screen displayed in Step S26 is almost the same as the ventilation amount calculation result screen 341 shown in FIG. 5, but in Step S26, the graph 341b shows temporal change in the ventilation amount of each lung field, and on the temporal change in the ventilation amount of each lung field, the mark 341c indicating the position of the time phase of the displayed frame image of the dynamic image 341a is displayed in sync with the dynamic image 341a.

Thus, the dynamic image 341a and the graph 341b are synchronized with each other and displayed in such a way as to be apposed. This allows a doctor(s) to observe the dynamic image 341a while checking the ventilation amounts of the right and left lung fields, and therefore can improve diagnostic efficiency.

In the above, one characteristic point is set on each of the right and left lung regions. Alternatively, a plurality of characteristic points may be set thereon. In this case, for each lung region, the displacement of each characteristic point is calculated, and the ventilation amount is calculated by substituting the mean of the displacements of the respective characteristic points for the “Displacement of Characteristic Point” in the above Formula 4. There is a case where the displacement of a characteristic point slightly differs according to the position of the characteristic point because of individual difference in body structure and/or imaging environment. Use of the mean of displacements of multiple characteristic points can absorb the difference in the displacement, so that the ventilation amount can be calculated with higher accuracy.

As described above, the hardware processor 31 of the diagnostic console 3 sets a characteristic point on a lung region in a dynamic image of the chest of a subject, measures a displacement of the characteristic point at each time phase of the dynamic image, obtains a reference ventilation amount of the subject, and calculates a ventilation amount at each time phase of the dynamic image on the basis of the reference ventilation amount and the displacement of the characteristic point at each time phase of the dynamic image.

Thus, the ventilation amount at each time phase of the dynamic image of the chest (i.e. dynamic chest image) is calculated on the basis of the displacement of the characteristic point at each time phase of the dynamic image. This can reduce influence of noise, so that the ventilation amount at each time phase can be calculated with high accuracy.

For example, the hardware processor 31 calculates a volume change amount of a lung field in the chest on the basis of the maximum displacement of the characteristic point in the dynamic image, and calculates the reference ventilation amount on the basis of the calculated volume change amount. Thereby, the reference ventilation amount can be obtained by imaging the dynamic state of the chest only, without using other modalities. Further, if the dynamic image contains a plurality of breathing cycles, the hardware processor 31 calculates the maximum displacement of the characteristic point for each of the breathing cycles, thereby obtaining the maximum displacements of the characteristic point in the respective breathing cycles, and calculates the reference ventilation amount on the basis of the mean of the maximum displacements in the respective breathing cycles. This can absorb variation in the ventilation amount among the breathing cycles, so that the ventilation amount can be calculated with high accuracy.

Further, for example, the hardware processor 31 obtains a ventilation amount of the subject measured with a spirometer as the reference ventilation amount. Thereby, the reference ventilation amount can be obtained with high accuracy.

Further, for example, the hardware processor 31 sets the characteristic point on the contour of the bottom part of the lung region in the dynamic image. Thereby, the ventilation amount can be calculated with high accuracy when the breathing style is mainly abdominal breathing.

Further, for example, the hardware processor 31 sets the characteristic point on the contour of the outer side part of the lung region in the dynamic image. Thereby, the ventilation amount can be calculated with high accuracy when the breathing style is mainly chest breathing.

Further, for example, if the hardware processor 31 sets, as the characteristic point, a plurality of characteristic points, the hardware processor 31 measures displacements of the respective characteristic points at each time phase of the dynamic image, and calculates the ventilation amount at each time phase of the dynamic image on the basis of the obtained reference ventilation amount and the mean of the displacements of the respective characteristic points at each time phase of the dynamic image. This can absorb, if the displacement of a characteristic point slightly differs according to the position of the characteristic point because of individual difference in body structure and/or imaging environment, the difference in the displacement, so that the ventilation amount can be calculated with high accuracy.

Further, for example, if the hardware processor 31 sets, as the characteristic point on the lung region, characteristic points on the right and left lung regions in the dynamic image, respectively, the hardware processor 31 measures displacements of the respective characteristic points on the right and left lung regions at each time phase of the dynamic image, obtains reference ventilation amounts of the right and left lung fields of the subject, respectively, and calculates ventilation amounts of the right and left lung fields at each time phase of the dynamic image, respectively, on the basis of the reference ventilation amounts of the right and left lung fields and the displacements of the characteristic points on the right and left lung fields at each time phase of the dynamic image. Thereby, the respective ventilation amounts of the right and left lungs (the right and left lung fields) at each time phase can be calculated with high accuracy.

Further, for example, the hardware processor 31 displays the dynamic image and a graph showing temporal change in the calculated ventilation amount on the display 34 such that the dynamic image and the graph are apposed, and displays a mark at a point for a displayed time phase of the dynamic image on the graph in sync with the dynamic image. This allows a doctor(s) to observe the dynamic image while checking the ventilation amount, and therefore can improve diagnostic efficiency.

Those described in the above embodiments are preferred examples of the dynamic analysis system of the present invention, and not intended to limit the present invention.

For example, in the above embodiments, the reference ventilation amount is calculated on the basis of the maximum displacement of the characteristic point, and the unit ventilation amount is obtained by dividing the reference ventilation amount by the maximum displacement. Alternatively, the reference ventilation amount may be calculated on the basis of the displacement of the characteristic point at an arbitrary time phase, and the unit ventilation amount may be obtained by dividing the reference ventilation amount by the displacement of the characteristic point at the arbitrary time phase.

Further, in the above embodiments, the ventilation amount is obtained by multiplying the unit ventilation amount by the displacement of the characteristic point. Alternatively, the ventilation amount may be obtained by multiplying the unit ventilation amount by the displacement of the characteristic point and further multiplying it by a coefficient.

Further, in the above embodiments, the relative distance from the position of the characteristic point at the maximal expiratory level serving as a reference to the position of the characteristic point at each time phase of the dynamic image is measured as the displacement of the characteristic point at each time phase of the dynamic image. Alternatively, if the characteristic point is set on the contour of the bottom part of the lung region in the dynamic image, the absolute distance from the apex of the lung region at each time phase of the dynamic image to the characteristic point at each time phase of the dynamic image may be measured as the displacement of the characteristic point at each time phase of the dynamic image. Further, a user may choose, to be measured as the displacement of the characteristic point, one of: the relative distance from the position of the characteristic point at a time phase of the dynamic image, the time phase serving as a reference; and the absolute distance from the apex of the lung region, by operating the operation unit 33. This allows a user(s) to choose an appropriate displacement, giving consideration to the correlation thereof with the actual ventilation amount.

Further, in the above embodiments, as the body thickness used for calculation of the reference ventilation amount, the body thickness that is stored in the storage 32 for the physical characteristics of the examinee is used. Alternatively, the body thickness calculated on the basis of the dynamic image may be used. For example, the chest of an examinee is imaged from the front and the side(s) so that dynamic images are obtained, and the width of the lung fields or the height of the lung fields is calculated from the dynamic chest image taken from the front, and the body thickness is calculated from the dynamic chest image(s) taken from the side(s).

Further, in the above, as a computer readable medium for the programs of the present invention, a hard disk, a nonvolatile semiconductor memory or the like is used. However, this is not a limit. As the computer readable medium, a portable recording/storage medium, such as a CD-ROM, can also be used. Further, as a medium to provide data of the programs of the present invention, a carrier wave can be used.

In addition to the above, detailed configurations and detailed actions of the components of the dynamic analysis system 100 can also be appropriately modified without departing from the spirit of the present invention.

Although embodiments of the present invention have been described and illustrated in detail, it is clearly understood that the same is by way of illustration and example only and is not to be taken by way of limitation, and the scope of the present invention should be interpreted by terms of the appended claims.

What is claimed is:

1. A dynamic analysis system that analyzes a dynamic image of at least one breathing cycle obtained by imaging a dynamic state of a chest of a subject, comprising:
a hardware processor that sets a characteristic point on a lung region in the dynamic image, measures a displacement of the characteristic point at each time phase of the dynamic image, obtains a reference ventilation amount of the subject, and calculates a ventilation amount at the each time phase based on the reference ventilation amount and the displacement of the characteristic point at the each time phase.

2. The dynamic analysis system according to claim 1, wherein the hardware processor calculates a volume change amount of a lung field in the chest based on a maximum displacement of the characteristic point in the dynamic image, and calculates the reference ventilation amount based on the calculated volume change amount.

3. The dynamic analysis system according to claim 2, wherein if the dynamic image contains a plurality of breathing cycles, the hardware processor calculates the maximum displacement of the characteristic point for each of the breathing cycles, thereby obtaining maximum displacements of the characteristic point in the respective breathing cycles, and calculates the reference ventilation amount based on a mean of the maximum displacements in the respective breathing cycles.

4. The dynamic analysis system according to claim 1, wherein the hardware processor obtains a ventilation amount of the subject measured with a spirometer as the reference ventilation amount.

5. The dynamic analysis system according to claim 1, wherein the hardware processor sets the characteristic point on a contour of a bottom part of the lung region in the dynamic image.

6. The dynamic analysis system according to claim 1, wherein the hardware processor sets the characteristic point on a contour of an outer side part of the lung region in the dynamic image.

7. The dynamic analysis system according to claim 1, wherein if the hardware processor sets, as the characteristic point, a plurality of characteristic points, the hardware processor measures displacements of the respective characteristic points at the each time phase of the dynamic image, and calculates the ventilation amount at the each time phase based on the reference ventilation amount and a mean of the displacements of the respective characteristic points at the each time phase.

8. The dynamic analysis system according to claim 1, wherein if the hardware processor sets, as the characteristic point on the lung region, characteristic points on right and left lung regions in the dynamic image, respectively, the hardware processor measures displacements of the respective characteristic points on the right and left lung regions at the each time phase of the dynamic image, obtains reference ventilation amounts of right and left lung fields of the subject, respectively, and calculates ventilation amounts of the right and left lung fields at the each time phase, respectively, based on the reference ventilation amounts of the right and left lung fields and the displacements of the characteristic points on the right and left lung fields at the each time phase.

9. The dynamic analysis system according to claim 1, further comprising an operation unit with which a user chooses one of a first distance and a second distance to be measured as the displacement of the characteristic point at the each time phase if the characteristic point is set on a contour of a bottom part of the lung region in the dynamic image, wherein
the first distance is a relative distance from a position of the characteristic point at a time phase of the dynamic image, the time phase serving as a reference, to a position of the characteristic point at the each time phase, and
the second distance is an absolute distance from an apex of the lung region at the each time phase to the characteristic point at the each time phase.

10. The dynamic analysis system according to claim 1, further comprising a display that displays the dynamic image and a graph showing temporal change in the calculated ventilation amount such that the dynamic image and the graph are apposed, and displays a mark at a point for a displayed time phase of the dynamic image on the graph in sync with the dynamic image.
